(19) United States
(12) Patent Application Publication
(10) Pub. No.: US 2023/0346892 A1
(43) Pub. Date: Nov. 2, 2023

(54) TREATMENT OF LONG BONE FRACTURES WITH ABALOPARATIDE

(71) Applicant: Radius Health, Inc., Boston, MA (US)

(72) Inventors: Beate Klara Maria Mannstadt, Arlington, MA (US); Bruce Mitlak, Boston, MA (US)

(73) Assignee: Radius Health, Inc., Boston, MA (US)

(21) Appl. No.: 18/346,426

(22) Filed: Jul. 3, 2023

Related U.S. Application Data
(63) Continuation of application No. PCT/IB2022/050048, filed on Jan. 4, 2022.
(60) Provisional application No. 63/134,027, filed on Jan. 5, 2021.

(51) Int. Cl. A61K 38/29; A61P 19/00

(52) U.S. Cl. CPC A61K 38/29; A61P 19/00

(57) ABSTRACT

Provided herein are methods of accelerating long bone fracture healing that include daily administration of a therapeutically effective amount of abaloparatide in combination with a surgical intervention for a long bone fracture. The daily administration is initiated prior to, at the time of, or following, surgical intervention for the long bone fracture and accelerates long bone fracture healing.

### RELATED APPLICATIONS

This application is a continuation of International Application No. PCT/IB2022/050048, filed Jan. 4, 2022 which claims priority to U.S. Provisional Patent Application 63/134,027, filed on Jan. 5, 2021, the contents of each are hereby incorporated by reference in their entirety.

### BACKGROUND OF THE INVENTION

In the United States, approximately 15 million bone fractures occur each year with over 500,000 of those including the tibia and fibula (Praemer, American Academy of Orthopaedic Surgeons, 1^{st} ed., 1992). Complication in fracture healing, such as delayed or non-union, is estimated to occur in 10% to 20% of normal civilian injuries. Co-morbidities including diabetes and aging can significantly increase rates of delayed union. Delayed healing and non-union are an even greater concerns for the military population than for civilians. Non-union rates as high as 50% have been reported one year after injury for open tibial fractures sustained during combat. Return-to-duty rates of only 22% are reported for US soldiers with isolated Type III open tibial fractures, which is less than half of the return-to-work rate for similarly severe injuries in the civilian population. For soldiers unable to return to duty, orthopedic injuries caused the greatest amount of permanent disability. These injuries also lead to significant emotional and psychiatric dysfunction and less than half of soldiers sustaining high-energy extremity trauma ever resume civilian employment (Doukas, J. Bone Joint Surg. Am. 2013 Jan. 16; 95(2):138-45). There is an unmet need for agents that accelerate or enhance long bone fracture healing.

### BRIEF SUMMARY OF THE INVENTION

Provided herein are methods of treating a subject having a long bone fracture that include daily administration of abaloparatide. Administration may follow a surgical intervention. Administration may also include pre-treatment with abaloparatide prior to surgery. Alternatively, in some embodiments, abaloparatide may be administered to avoid surgical intervention due to non-union.

In one aspect, a method of accelerating or enhancing long bone fracture healing in a subject in need thereof is provided. The method includes administering a therapeutically effective amount of abaloparatide to the subject following surgical intervention for the long bone fracture. In some embodiments administration begins 2 weeks after surgical intervention. Additionally, in some embodiments, administration is daily administration for 3 months.

In some embodiments, the long bone fracture is a tibia fracture. In others, the long bone fracture is a fracture of the femur, humerus, radius, ulna, fibula, metatarsus, phalanges, metacarpus, or clavicle. In some embodiments, there are multiple long bone fractures, as in the case of a multiple trauma.

In some embodiments, accelerated or enhanced healing is evidenced by fracture healing, callus formation, or both, confirmed at 6, 12, or 24 weeks by least one of: CT scan, bone mineral density, 3D volume of callus formation, fracture site stiffness by Dynamic Stereo X-Ray (DSX), and modified Radiographic Union Score for Tibial Fractures (mRUST) assessment for fracture healing. In some embodiments, union is accelerated, as determined by a mRUST of greater than >13 at 6 weeks, 12 weeks, or 24 weeks. In some embodiments, bone quality is confirmed by a bone density scan (qCT) at 12 weeks, or 24 weeks, or both. In some embodiments, cartilaginous phase of fracture repair is confirmed by detection of circulating levels of collagen X (“CXM”). In some embodiments, fracture site stiffness is achieved at 6 weeks or 12 weeks.

In some embodiments, administering the therapeutically effective amount of abaloparatide elevates bone formation markers without elevating bone resorption markers.

In some embodiments, the surgical intervention is selected from internal fixation, external fixation, intramedullary nailing, a bone graft, a prosthetic, or a combination thereof. In some embodiments, the surgical intervention is an implantation of an intramedullary (IM) rod.

In some embodiments, the administration is daily subcutaneous administration of from about 20 to about 100 μg of abaloparatide. In some embodiments, the administration is daily subcutaneous administration of 80 μg abaloparatide.

In some embodiments, the administration is daily transdermal administration of from about 100 to about 400 μg of abaloparatide. In some embodiments, the administration is daily transdermal administration of 300 μg of abaloparatide.

In some embodiments, the abaloparatide is administered daily for at least 2 months, 3 months, 4 months, 6 months, 12 months or 18 months. In some embodiments, the abaloparatide is administered daily for 3 months.

In some embodiments, the method further includes pre-treating by administering a therapeutically effective amount of abaloparatide to the subject prior to the surgical intervention.

In some aspects, a method of accelerating, promoting or enhancing long bone fracture healing in a subject in need thereof is provided. The method includes administering a therapeutically effective amount of abaloparatide to the subject daily for 3 months following surgical intervention for the long bone fracture. In some embodiments, the administration is daily subcutaneous administration of 80 μg of abaloparatide. In some embodiments, the administration is daily transdermal administration of 300 μg of abaloparatide.

In some embodiments of any of the above methods, the subject is at high risk for fractures. In some embodiments, the high risk is attributable to smoking, diabetes, vascular disease, or combinations thereof.

In some embodiments, the healing time is decreased by at least 25%, as compared to a subject who has not received abaloparatide therapy. In some embodiments, healing time is shortened by 2 weeks, 6 weeks, 2 months, or even 6 months, as compared to healing without administration of abaloparatide.

### DETAILED DESCRIPTION OF THE INVENTION

While there is no single consensus around the definition of a non-union fracture, the United States Food and Drug Administration (FDA) guidelines suggest 9 months without radiographic evidence of bone healing before treatment. Non-union fractures are up to 97 times more likely to require surgical intervention to promote healing. Provided herein are methods of accelerating the healing rate of subjects with long bone fractures by administering abaloparatide.

Abaloparatide is currently approved for the treatment of postmenopausal women with osteoporosis at high risk for fracture. Preclinical studies have indicated improved fracture healing in rats with internally fixed femur fractures (Lanske, et al. “Abaloparatide, a PTH receptor agonist with homology to PTHrP, enhances callus bridging and biomechanical properties in rats with femoral fracture,” J Orthop Res. 2019 April; 37(4):812-820). Recent clinical studies comparing the efficacy of abaloparatide over teriparatide have demonstrated greater bone mineral density increases and less hypercalcemia in treating osteoporosis (Sleeman, Am J Health Syst Pharm 76, 130-135 (2019); Miller, JAMA 316, 722-733 (2016)). While some studies have been conducted to evaluate the efficacy of teriparatide in fracture healing (Shi, et al., Effectiveness of Teriparatide on Fracture Healing: A Systemic Review and Meta-Analysis, PLOS ONE, 2016: e0168691; Roberts et al. Anabolic Strategies to Augment Bone Fracture Healing. Curr Osteoporos Rep. 2018; 16(3):289-298), the study described herein below in Example 1 will be the first clinical study testing efficacy of abaloparatide in fracture healing. Due to the relatively high degree of interfragmentary motion (IFM) in tibia fractures, these long bone injuries heal predominantly through endochondral ossification, where a cartilage intermediate drives secondary bone formation. Without wishing to be bound to any particular theory, it is believed that abaloparatide could be effective in treating these fractures in humans because abaloparatide binds to the PTH1 G-coupled protein receptor, which is highly expressed on both chondrocytes and osteoblasts, and therefore will impact both the endochondral and intramembranous phases of fracture repair.

Example 1 presents a double-blinded and randomized fracture healing trial in which patients with tibia fractures treated with an intramedullary rod are randomized into abaloparatide versus placebo therapy. Tibia fracture healing was chosen as the primary long bone fracture of study because the modified Radiographic Union Score for Tibia (mRUST) fracture cortical scoring system is validated for the grading of callus formation and radiographic progression towards union in tibial diaphyseal fractures with intraclass correlation coefficients (ICCs) of 0.74-0.89 reported (Mitchell et al., J Orthop Trauma. 2019 June;33(6):301-307; Fiset, et al., J Bone Joint Surg Am. 2018 Nov. 7; 100(21):1871-1878; Litrenta et al., 2015 November;29(11):516-20). In addition, a fracture biomarker assay can be employed to demonstrate early bone healing (Working et al., Journal of Orthopaedic Research, 13 Jun. 2020; Coghlan et al., Sci Transl Med 2017 December 6; 9:419), as well as advanced dynamic imaging (Tashman, S, Journal of Biomechanical Engineering. 2003 April;125(2):238-245) and biomechanical assessments of repair quality. A new serum-based biomarker that measures the cartilaginous phase of fracture repair through detection of circulating levels of collagen X (“CXM”) is also employed, as it is suspected according to the present disclosure that abaloparatide will work mechanistically to improve the endochondral phase of fracture healing. It is believed that the measurements of tibia healing (CXM biomarker, dynamic imaging, biomechanics) in the Example 1 study will detect meaningful changes in the rate of fracture healing following administration of abaloparatide.

Provided herein are methods of enhancing or accelerating long bone fracture healing in a subject in need thereof that include administering a therapeutically effective amount of abaloparatide to the subject. In some embodiments, administration follows surgical intervention, e.g., two weeks after surgical intervention for the long bone fracture. In some embodiments, administration of abaloparatide may be used to prevent the need for surgical intervention. In some embodiments, administration of abaloparatide may begin prior to, or during, the surgical intervention. The long bone fracture may be a fracture of the tibia. Alternatively, or additionally, e.g., in the case of a trauma, the long bone fracture may be one or more fractures in a clavicle, humerus, radius, ulna, metacarpus, phalange, femur, fibula, and/or metatarsus.

As used herein, the terms “accelerating,”, “accelerate,” “enhancing,” “enhanced,” “enhance,” and the like, refer to intensifying, accelerating, or amplifying the quality, value, or extent of long bone fracture healing, relative to healing without administration of abaloparatide.

Depending on the type of long bone fracture (e.g., transverse, oblique, spiral or comminuted), and the severity and number of breaks or splinters, there are several surgical interventions that can be employed. They are generally grouped into three categories: internal fixation, external fixation, and intramedullary nailing. For internal fixation, metal pins, plates, screws or the like, are placed into the bone to create and maintain alignment of the fractured bone. For external fixation, metal pins or screws are placed into the bone above and below the site of the fracture, which is then connected to a bar or mesh outside of the bone. In intramedullary nailing, a metal rod is inserted into the center of the bone to keep the bone aligned and stable. Recovery time may be 4-6 months to completely heal, or it may take 9 months or longer depending on the fracture and the age and health of the patient. In some embodiments the human subject does not have osteoporosis.

In some embodiments abaloparatide is delivered via a subcutaneous injection that delivers 80 μg abaloparatide daily, e.g., the device and formulation for the currently approved TYMLOS abaloparatide injection product. In some embodiments, the device and formulation is disclosed in U.S. Pat. Nos. 7,803,770, issued Sep. 28, 2010; U.S. Pat. No. 8,148,333, issued Apr. 3, 2012; and U.S. Pat. No. 8,748,382, issued Jun. 10, 2014, all of which are expressly incorporated by reference in their entirety.

In some embodiments, the abaloparatide is delivered transdermally. In some embodiments, the transdermal device is a device, applicator and/or formulation disclosed in International Patent Application Publication Nos. WO2017/062922, published 13 Apr. 2017; WO2017/184355, published 26 Oct. 2017; WO2017/062727, published 13 Apr. 2017; WO2017/184355, published 26 Oct. 2017; WO2019/077519, published Apr. 24, 2019; and WO2020/17443, published Sep. 30, 2020, all of which are expressly incorporated by reference in their entirety. These transdermal devices can be deployed with a single-use applicator or an application capable of being used multiple times. In some embodiments, the abaloparatide formulation includes zinc salts (e.g., ZnCl₂). In some embodiments, the transdermal device is a transdermal patch that delivers about 300 μg of abaloparatide, and the transdermal patch is administered daily.

In some embodiments, the healing time in a subject treated with abaloparatide is decreased by at least 10%, at least 20%, at least 25%, at least 30%, at least 40%, or at least 50%, compared to the healing time in an untreated subject of the same age group, undergoing a similar surgical procedure and having the same type of fracture. In some embodiments, the healing time in a subject treated with abaloparatide is decreased by at least 2 weeks, 4 weeks, 6 weeks, 8 weeks, 3 months, 6 months, a year, or 18 months, compared to the healing time in an untreated subject of the same age group, undergoing a similar surgical procedure and having the same type of fracture. In some embodiments, the degree of decrease in fracture healing time in subjects treated with abaloparatide, compared to the untreated subjects, correlates with their age. For example, the highest decrease in fracture healing time may be observed in the oldest subjects treated with abaloparatide. In certain embodiments, the percentage decrease in fracture healing time is higher in older subjects. In some embodiments, the percentage decrease in healing time in older subjects is higher by at least 10%, at least 20%, at least 30%, at least 40%, or at least 50%, compared to the percentage decrease in healing time in younger subjects having the same type of fracture.

There are generally three stages of fracture healing: the formation of hematoma or inflammatory phase, a repair phase where there is a formation of a cartilage and fibrous callus, and a remodeling phase where the callus is replaced with solid bone.

In some embodiments, callus formation is achieved in 6 weeks, 12 weeks or 24 weeks. In some embodiments, fracture healing is achieved in 6 weeks or 12 weeks. In some embodiments, fracture healing is achieved in 6 months. In some embodiments, the rate or chance of non-union is less than 25%. In some embodiments, the chance or rate of non-union is less than 10%. In some embodiments, chance or rate of non-union is less than 5%.

Fracture healing and callus formation may be confirmed by CT scan, bone mineral density, and/or 3D volume of callus formation. Fracture healing and callus formation may be confirmed by fracture site stiffness at 6 weeks, 12 weeks and/or 24 weeks by Dynamic Stereo X-Ray (DSX). Fracture healing may be confirmed by mRUST radiographic assessment for assessing fracture healing. In certain embodiments the mRUST score is greater than 13.

In any of these embodiments, the therapeutically effective amount is clinically proven to be effective in humans as demonstrated in a clinical trial. The terms “clinical efficacy,” “clinically effective,” and the like, refer to efficacy as demonstrated in a clinical trial conducted by the US Food and Drug Administration (FDA), or any foreign counterpart, e.g., the European Medicines Agency.

Also provided is a method of long bone fracture healing in a subject in need thereof, the method comprising administering a therapeutically effective amount of abaloparatide to the subject and wherein surgical intervention is not required for non-union.

The exemplification is provided to better illustrate the claimed methods and are not to be interpreted as limiting the scope of the methods provided herein and claimed. To the extent that specifics are mentioned, it is for purposes of illustration and is not intended to limit the disclosure. It will be understood that many variations can be made in the procedures herein described while still remaining within the bounds of the present disclosure. It is the intention of the inventors that such variations are included within the scope of the disclosure.

### EXEMPLIFICATION

### Example 1: Abaloparatide Therapy to Accelerate Tibia Fracture Healing

This is the first clinical intervention with abaloparatide aimed at accelerating tibia fracture healing. This study is a double blind, randomized 2-arm, study of tibia fractures treated with an intramedullary (IM) rod. Following surgery, patients will be randomized into two groups: tibia fractures treated with placebo, and tibia fractures treated with abaloparatide. There will be 30 subjects per arm, 60 subjects total. The duration of this study is expected to be about 3 years for 60 patients at 24 weeks per patient.

Placebo or abaloparatide therapy will begin 2 weeks post-operatively and continue for 3 consecutive months of daily administration at a dose of 80 μg. All groups will receive either a placebo or abaloparatide injection pens and the appearance of the pens will be identical. If subjects experience common side effects (headache or dizziness), they will have their daily injection dosage reduced to 40 μg for two weeks, and then resume the full 80 μg dosage. The complete dosage of the drug will be added to the conclusion of the study.

Patients will return for follow-up evaluations of healing at 2, 6, 12, 18, and 24 weeks or until union is achieved or malunion diagnosed (6 months). The primary endpoint for this study is time to mRUST score of >13. Secondary healing assessments will include the serum-based collagen X biomarker (“CXM”), advanced dynamic imaging and quantitative CT scans, and standardized patient-reported outcomes.

Eight small (1 mm) tantalum beads (four proximal and four distal to the fracture) will be implanted into the tibia of participants at time of surgery to provide well defined radiopaque landmarks. This will allow researchers to perform a full 3D kinematic analysis using high-speed biplane radiography.

### Inclusion Criteria

Patients will be: male or female; age 18 years or older; have a minimum of community ambulator prior to injury; have open or closed diaphyseal tibial fractures (AO/OTA type 42); open fractures must be Gustilo-Anderson type 1, 2 or 3A without significant periosteal damage; scheduled to receive surgical treatment for unilateral tibial diaphyseal fracture; and have a planned internal tibia fixation with intramedullary (IM) rods (reamed/unreamed, locked/unlocked).

### Exclusion Criteria

Excluded from the study will be patients that have/had: hypercalcemia or hyperparathyroidism; Paget's Disease or any other bone diseases; bone cancer; high alkaline phosphatase levels in the blood; fracture within the last year; retained tibial implants; concomitant injuries or other fractures that would significantly inhibit mobility or lower extremity function; additional fractures due to polytrauma; significant wound infection; history of significant radiation exposure (e.g. for cancer therapy); Wolff-Parkinson-White syndrome; chronic, well-established and medically supported hypertension with a causational history of fainting; women who are pregnant or plan to become pregnant over the duration of the study and women who are breastfeeding; have a BMI>35; have taken abaloparatide previously for the lifetime maximum (2 years), or will surpass the lifetime maximum over the course of the study.

### CT Scan (12, 24-Weeks)

Post-surgery, a 12-week CT scan will be obtained to determine bone/fracture geometry, implanted marker location and early callus formation. A 24-week CT will also be acquired as a final quantitative assessment of healing. Besides qualitative assessment of healing, bone mineral density (BMD) and the 3D volume of callus formation will be quantified. A bone density calibration phantom will be placed underneath the leg during the scans for density calibration.

### Venipuncture for Fracture Biomarker and Metabolic Safety Panel (0, 2, 6, 12, 18, 24 Weeks)

Blood (10 mL) will be collected initially and at each follow-up visit (0/baseline, 2, 6, 12, 18, and 24 weeks). Five drops of blood will be transferred from the collection syringe or tubing to a 903 Protein Saver Card, 3 mL will be set aside for Vail Health Hospital Lab to run the metabolic panel and Bone Specific Alkaline Phosphatase (BSALP) levels. These are precautionary safety screenings to ensure no adverse effects take place due to the drugs associated risks of hypercalcemia and elevated ALP and BSALP levels. The additional blood will be further processed via centrifugation to collect the supernatant/serum. The serum will be transferred to cryovials, labeled with the data, volume of sample and de-identified patient indicator and then at −80C. Biomarker data will be stored until entire study is completed and then sent for quantification using a validated ELISA-based bioassay.

### Patient Reported Outcome (PRO) Assessment (0, 2, 6, 12, 18, 24 Weeks)

A customized electronic questionnaire will be compiled to capture several standard PRO scales. PRO scores will be calculated and stored in a secure MySQL database. PROs include BPI, SFMA, VR-12, PHQ-9, and PSQ-18. A fracture questionnaire will also be given at the first venipuncture.

### Dynamic Stereo X-Ray (DSX) Imaging (6, 12, 24 Weeks)

DSX provides a dynamic, three-dimensional assessment of motion between the fracture fragments. The lab is equipped with an 18-camera 3D video-motion analysis system, a dual-belt instrumented treadmill, 4 force plates and a DSX imaging system (all time-synchronized). The x-ray generators can produce 1 ms pulses at rates up to 250 images/s, providing low-dose, blur-free images. Image detection is provided by 43-cm flat-panel detectors (FPD's) capable of 3072×3072 pixel resolution at 30 frames/s (or 1536×1536 pixels at 60 frames/s) with 14-bit dynamic range. Previous dynamic studies achieved precision of better than 70 μm for tracking similar bone-implanted markers using low-resolution (512×512 pixel) image intensifiers. With pixels of ⅓ to ⅙th the size, we expect to achieve accuracy in the 25-50 μm range. Data will be collected for 3 trials each of non-weight bearing to weight bearing transition and treadmill gait (1 m/s, as tolerated). 3D coordinates of implanted markers will be determined from the biplane images using radio-stereophotogrammetric techniques and smoothed using a 25-Hz 4th order, zero lag Butterworth low pass filter. Transformations between marker-based coordinates and anatomical axes will determined from high-resolution CT scans. Rotations of the distal tibial fragment relative to the proximal fragment will be calculated using body fixed axes in the order of sagittal, coronal, and transverse planes. Displacements will be measured between the centroids of the distal end of the proximal fragment and the proximal end of the distal fragment (identified by CT), and expressed in a coordinate system fixed to the proximal fragment. Peak axial, shear motion, and rotational/angular displacements between the tibial bone fragments will be determined and averaged across gait cycles. Fracture site stiffness will be estimated by dividing the net force applied to the distal tibia (determined via inverse dynamics using Visual 3D; C-Motion) by the angular and linear displacement of the fracture in each plane.

### Activity Monitoring (Daily)

Participants will record their average steps per day, and provide their daily logs at each subsequent visit (6, 12, and 24 weeks).

mRUST RADIOGRAPHS

The modified RUST (mRUST) radiographic assessment will be used for assessing fracture healing. Using the DSX system, one standing combined A/P and lateral radiographs, will be acquired prior to DSX dynamic testing. Longer exposures (8 ms, compared to 1 ms for dynamic imaging) will be used to provide diagnostic-quality images. mRUST scores each cortex on AP and lateral radiographed as 1=no callus, 2=callus present, 3=bridging callus, 4=remodeled, fracture not visible. The modified RUST score is the sum of these and therefore has a value from 4 to 16. Time to bridge 1 to 3 cortices will be determined from the mRUST radiographs by at least two blinded reviewers.

The primary endpoint for this study is time to mRUST score of >13, with secondary healing assessments (including CT scan, DSX imaging, and patient reported outcomes) scoring better in the treatment group than that of the placebo group.

1. A method of accelerating long bone fracture healing in a subject in need thereof, the method comprising administering a therapeutically effective amount of abaloparatide to the subject following surgical intervention for the long bone fracture.

2. The method according to claim 1, wherein the long bone fracture is a tibia fracture.

3. The method according to claim 1, wherein fracture healing, callus formation, or both, is confirmed at 6, 12, or 24 weeks by at least one of: CT scan, bone mineral density, 3D volume of callus formation, fracture site stiffness by Dynamic Stereo X-Ray (DSX), and mRUST radiographic assessment for assessing fracture healing.

4. The method according to claim 1, wherein union is accelerated, as determined by a modified Radiographic Union Score for Tibial Fractures (mRUST) of greater than 13 at 6 weeks, 12 weeks, or 24 weeks.

5. The method according to claim 1, wherein bone quality is confirmed by a bone density scan (qCT) at 12 weeks, or 24 weeks, or both.

6. The method according to claim 1, wherein cartilaginous phase of fracture repair is confirmed by detection of circulating levels of collagen X (“CXM”).

7. The method according to claim 1, wherein fracture site stiffness is achieved at 6 weeks or 12 weeks.

8. The method according to claim 1, wherein administering the therapeutically effective amount of abaloparatide elevates bone formation markers without elevating bone resorption markers.

9. The method according to claim 1, wherein the surgical intervention is selected from: internal fixation, external fixation, intramedullary nailing, a bone graft, a prosthetic, or combination thereof.

10. The method according to claim 1, wherein the surgical intervention is an implantation of an intramedullary (IM) rod.

11. The method according to claim 1, wherein the administration is daily subcutaneous administration of from about 20 to about 100 μg abaloparatide.

12. The method according to claim 11, wherein the administration is daily subcutaneous administration of 80 μg of abaloparatide.

13. The method according to claim 1, wherein the administration is daily transdermal administration of from about 100 to about 400 μg of abaloparatide.

14. The method according to claim 13, wherein the administration is daily transdermal administration of 300 μg of abaloparatide.

15. The method according to claim 1, wherein the abaloparatide is administered daily for at least 2 months, 3 months, 4 months, 6 months, 12 months or 18 months.

16. The method according to claim 15, wherein the abaloparatide is administered daily for 3 months.

17. The method according to claim 1, further comprising pre-treating by administering a therapeutically effective amount of abaloparatide to the subject prior to the surgical intervention.

18. A method of accelerating long bone fracture healing in a subject in need thereof, the method comprising administering a therapeutically effective amount of abaloparatide to the subject daily for 3 months following surgical intervention for the long bone fracture.

19. The method according to claim 18, wherein the administration is daily subcutaneous administration of 80 μg of abaloparatide.

20. The method according to claim 18, wherein the administration is daily transdermal administration of 300 μg of abaloparatide.

21. The method according to claim 1, wherein the subject is at high risk for fractures.

22. The method according to claim 21, wherein the high risk is attributable to smoking, diabetes, vascular disease, or combinations thereof.

23. The method according to claim 1, wherein the healing time is decreased by at least 25%, as compared to a subject who has not received abaloparatide therapy.

24. The method according to claim 18, wherein the subject is at high risk for fractures.

25. The method according to claim 18, wherein the healing time is decreased by at least 25%, as compared to a subject who has not received abaloparatide therapy.
